SEQUENCE LISTING (14 sequences)

SEQ ID NO: 1 - DNA, 25 bp, synthetic construct
  source 1..25: mol_type = other DNA
caggtgtgattgatagtaaacttac

SEQ ID NO: 2 - DNA, 25 bp, synthetic construct
  source 1..25: mol_type = other DNA
gattgatagtaaacttacttatatc

SEQ ID NO: 3 - DNA, 25 bp, synthetic construct
  source 1..25: mol_type = other DNA
gtaaacttacttatatctcaagtac

SEQ ID NO: 4 - DNA, 25 bp, synthetic construct
  source 1..25: mol_type = other DNA
caggtgtgattgatagtaaccttac

SEQ ID NO: 5 - DNA, 25 bp, synthetic construct
  source 1..25: mol_type = other DNA
gtaaccttacttacatctcaagtac

SEQ ID NO: 6 - DNA, 25 bp, synthetic construct
  source 1..25: mol_type = other DNA
gtaaccttacttatatctcaagtac

SEQ ID NO: 7 - DNA, 25 bp, synthetic construct
  source 1..25: mol_type = other DNA
tatctcaagtacataggattctctg

SEQ ID NO: 8 - DNA, 25 bp, synthetic construct
  source 1..25: mol_type = other DNA
gatagtaaccttacttatatctcaa

SEQ ID NO: 9 - DNA, 21 bp, synthetic construct
  source 1..21: mol_type = other DNA
caggtgtgattgatagtaaac

SEQ ID NO: 10 - DNA, 25 bp, synthetic construct
  source 1..25: mol_type = other DNA
catgtaattgttggtacttctgtaa

SEQ ID NO: 11 - DNA, 25 bp, synthetic construct
  source 1..25: mol_type = other DNA
tcttcccaagaggcccacctataag

SEQ ID NO: 12 - DNA, 25 bp, synthetic construct
  source 1..25: mol_type = other DNA
cacctgtggtatcactccaaaggct

SEQ ID NO: 13 - DNA, 25 bp, synthetic construct
  source 1..25: mol_type = other DNA
ggctaagtccttttgctcacctgtg

SEQ ID NO: 14 - protein, 14 aa, synthetic construct
  MOD_RES 14: note = beta-alanine
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
  MOD_RES 13: note = 6-aminohexanoic acid
RKRRKRRKRRKRKA

(19) United States
(12) Patent Application Publication
(10) Pub. No.: US 2026/0109986 A1
(43) Pub. Date: Apr. 23, 2026

(54) THERAPEUTIC OLIGONUCLEOTIDES TO CORRECT CYSTIC FIBROSIS MUTATIONS

(71) Applicant: The University of North Carolina at Chapel Hill, Chapel Hill, NC (US)

(72) Inventors: Silvia Kreda, Mebane, NC (US); Yan Dang, Chapel Hill, NC (US); Aiguo Ni, Chapel Hill, NC (US)

(21) Appl. No.: 19/117,758

(22) PCT Filed: Oct. 13, 2023
(86) PCT No.: PCT/US2023/076868

§ 371 Date: Apr. 2, 2025

Related U.S. Application Data
(60) Provisional application No. 63/379,591, filed on Oct. 14, 2022.

(51) Int. Cl. C12N 15/113; A61K 31/712; A61K 31/7125; A61K 45/06; A61K 48/00; A61P 11/00

(52) U.S. Cl. CPC C12N 15/1138; A61K 31/712; A61K 31/7125; A61K 45/06; A61K 48/005; A61P 11/00; C12N 2310/11; C12N 2310/31; C12N 2310/321; C12N 2310/3233; C12N 2310/3513; C12N 2320/33

(57) ABSTRACT

This invention relates to the finding that novel splice switching oligonucleotides can correct splicing mutations. Moreover, the invention relates to using the novel splice switching oligonucleotides to correct a 1811+1.6kb G>T (c. 1679+1643G>T). 1811+1.6kb A>G (c. 1679+1634A>G), 1811+1.6kb T>A (c. 1679+1650T>A), or W1282X (c.3846G>A) mutation in a pre-mRNA produced from the human CFTR gene and methods of using the same for treatment of cystic fibrosis (CF) in a subject.

### STATEMENT OF PRIORITY

This application claims the benefit, under 35 U.S.C. § 119(e), of U.S. Provisional Application No. 63/379,591, filed Oct. 14, 2022, the entire contents of which are incorporated by reference herein in their entirety.

### STATEMENT OF GOVERNMENT SUPPORT

This invention was made with government support under Grant No. TR002692 awarded by the National Institutes of Health. The government has certain rights in the invention.

### STATEMENT REGARDING ELECTRONIC FILING OF A SEQUENCE LISTING

A Sequence Listing in XML format, entitled 5470-940WO_ST26.xml, 15,584 bytes in size, generated on Oct. 13, 2023 and filed herewith, is hereby incorporated by reference in its entirety for its disclosures.

### FIELD OF THE INVENTION

This invention relates to oligonucleotides for delivery to a subject and methods of using the same for treatment of cystic fibrosis (CF) in a subject.

### BACKGROUND OF THE INVENTION

Ten percent or more of people with CF (pwCF) cannot benefit therapeutically from highly effective modulator treatments (HEMT) because of reduced cystic fibrosis transmembrane conductance regulator (CFTR) mRNA and protein levels resulting from severe splicing or nonsense CFTR mutations. Oligonucleotide therapies targeting these mutations constitute an attractive therapeutic strategy because of their efficacy and the fact that the bench-to-bed development track for oligonucleotide agents is fast compared to small molecules and other nucleic acid-based therapeutics. Notably, the past 5 years have seen the first clinical successes for oligonucleotide therapeutics in rare diseases.

RNA splicing involves the precise removal of introns from precursor (pre) messenger RNA (mRNA) to create mature mRNA assembled into translational reading frames. It requires conserved, canonical regulatory sequences at the exon-intron junctions (donor-acceptor sites) and at other exon and intronic locations. These sequences are recognized by the splicing machinery. Splicing mutations eliminate/create these recognition sequences, causing the insertion of non-exonic or elimination of exonic sequences in the mature mRNA. Splicing is a dynamic process and numerous factors, including age and inflammation, produce spatial-temporal specific splicing patterns for each gene.

In CF, 15-30% of disease-causing mutations are splicing errors. It is not surprising that CF, like other disorders caused by splicing errors, shows variability in disease severity and among different tissues in the same patient. The most frequent CFTR splicing mutation, 3849+10kbC>T, alters CFTR normal splicing and the production of functional CFTR protein. Severe splicing mutations in the CFTR gene typically result in little or no functional protein, reduced channel activity, severe CF disease phenotypes, and resistance to HEMT because of the low CFTR protein levels. Splicing mutations CFTR 1811+1.6 kb G>T, 1811+1.6 kb A>G, and 1811+1.6 kb T>A are three different point mutations in the 12th intron that create new splicing sites and allow for the insertion of an intronic fragment in the coding sequence; the result is a non-productive mRNA, low levels of CFTR protein, and severe CF disease phenotypes. Similarly, W1282X is a nonsense mutation in exon 23 and produces usually no CFTR protein.

There is a need in the art for a viable therapeutic approach for CF patients that is based on the use of antisense oligonucleotides (ASOs).

### SUMMARY OF THE INVENTION

Recently published oligonucleotide technology, with improved delivery to extra-hepatic tissues, can efficiently correct splicing mutations in the lungs and intestines of mice. Moreover, full correction of the CFTR 3849-10kbC>T splicing mutation was shown with a one-time treatment of this oligonucleotide technology in CF patient-derived human bronchial epithelial cells (HBEC). The results demonstrated production of full-length CFTR mRNA and improved channel activity after oligonucleotide treatment. These studies suggest that oligonucleotide technology is a stand-alone therapeutic strategy to correct many CFTR splicing mutations, such as 3849+10kbC>T.

This invention is based on the finding that novel splice switching oligonucleotides can correct a splicing mutation in CFTR and thereby treat CF.

First, splice switching oligonucleotides (SSOs) are utilized that can be chemically modified to improve bioavailability or efficacy, e.g., peptide-morpholino oligomer conjugates (PPMOs). These molecules have a broad tissue distribution and, when given systemically, the SSOs can produce oligonucleotide effects in extra-hepatic tissues. Second, oligonucleotide endosomal compounds (OECs) can also be used. These are small molecules, discovered through high throughput screening, which selectively release oligonucleotides from non-productive entrapment in endosomal compartments. Thus, OECs allow oligonucleotides to access the cytosol and nucleus providing substantial enhancement of pharmacological effects. Herein, we describe use of novel SSOs, with and without OECs, to efficiently correct a splicing defect in CFTR in CF patient-derived HBECs and other cells or tissues comprising an important CFTR splicing mutation.

Accordingly, one aspect of the invention is a splice switching oligonucleotide for correcting a 1811+1.6 kb G>T (c.1679+1643G>T), 1811+1.6 kb A>G (c.1679+1634A>G), 1811+1.6 kb T>A (c.1679+1650T>A), or W1282X (c.3846G>A) mutation in the pre-mRNA produced from the human CFTR gene, wherein the oligonucleotide specifically hybridizes to an mRNA produced from the mutated CFTR gene at a site within 100 nucleotides of the mutation, optionally within 25 nucleotides of the mutation, optionally comprising at least 5 consecutive nucleotides of any one of SEQ ID NOS: 1-13, optionally comprising a sequence at least 70% identical to any one of SEQ ID NOS: 1-13, or optionally comprising a sequence identical to any one of SEQ ID NOS: 1-13.

In some embodiments, the SSO has one or more modifications (e.g., 2′-O-methoxyethyl, 5′ constrained ethyl, phosphorothioate, phosphorodiamidate morpholino).

In some embodiments, the SSO is conjugated to a peptide, optionally a cationic peptide, optionally a peptide that is at least 90% identical to RXRRXRRXRRXRXB (SEQ ID NO:14), wherein R is arginine, B is β-alanine, and X is 6-aminohexanoic acid.

One aspect of the invention is a pharmaceutical composition comprising the SSO in a pharmaceutically acceptable carrier, optionally including an oligonucleotide endosomal compound.

One aspect of the invention is a method for correcting a 1811+1.6 kb G>T (c. 1679+1643G>T), 1811+1.6 kb A>G (c.1679+1634A>G), 1811+1.6 kb T>A (c. 1679+1650T>A), or W1282X (c.3846G>A) mutation in the pre-mRNA produced from the CFTR gene in a cell, optionally in a subject, comprising administering the SSOs of the invention.

Another aspect of the invention is a method of treating or delaying the onset of CF in a subject, comprising administering the SSOs of the invention.

These and other aspects of the invention are set forth in more detail in the description of the invention below.

### BRIEF DESCRIPTION OF THE DRAWINGS

FIGS. 1A-1C show that CFTR activity is restored using SSOs in HBEC from a 3849+10kbC>T homozygous CF patient. (A) Schematics of the 3849+10kbC>T splicing mutation and SSO correction. The intronic splicing mutation, denoted by an X, creates a new splicing site and elicits the incorporation in the coding sequence of 89 bp of intronic sequence as a pseudo-exon (Y′, orange box); there are downstream stop codons in-frame which can affect the stability of mRNA and/or the activity of the protein. Although read-through and production of WT mRNA and protein are also observed in some pwCF, a sequence-specific SSO can modulate the splicing machinery and elicit “correct” mRNA splicing and the synthesis of functional CFTR protein. (B,C) Three CFTR-targeted and a non-CFTR-targeted (MM623) SSOs were electroporated (50 nM) into primary bronchial cells from a subject homozygous for the 3849+10kbC>T mutation, which were then differentiated for 14 days and subjected to (B) PCR analysis: showing the lower band (wild type, WT) is the correctly spliced CFTR, while the mutant is the top band due to an 89 bp intronic insertion, and (C) Ussing chamber analysis showing ˜100% functional correction; TERs ˜700 Ω/cm2 in all experimental groups; n=3. These cells do not respond to Trikafta®.

FIGS. 2A-2B are a series of confocal microscopy images showing the oligonucleotide delivery in HBEC. (A) OEC increases the delivery of a fluorescent-oligo (50 nM, 7 hours) to the nuclei of epithelial cells (cilia stained with a fluorescent-lectin). (B) Fluorescent-PPMO (0.5 UM, 1 hour) do not require OEC for nuclear delivery; PPMO signal in the nuclei of HBEC is detected >6 months after one PPMO treatment. Live cell imaging using xz (A) and xy (B) confocal microscopy scanning (Leica SP5, 63X lens).

FIGS. 3A-3E show that CFTR activity is fully restored with the present oligonucleotide technology (peptide-morpholino), but not with HEMT (Trikafta® equivalent) in HBEC from a 3849+10kbC-T homozygous CF patient. Passage 2 CF (primary) HBEC were treated one time with PPMO3849 (overnight, 1 μM) and OEC UNC7938 (2 hours, 10 μM). PPMO & OEC (P&O) were added basolaterally (BL) or apically (AP, 30 μl). The following day, vehicle or Trikafta®-like (Tr=VX-445, 2 μM, VX-661, 3 μM & VX-770, 1 μM; 48 hours) were added BL+AP. All drugs were added in medium. (A-C) Analysis of CFTR functional correction (Ussing chambers): representative trace diagram (A); post-amiloride forskolin short circuit current peak values (B) and transepithelial resistances (TER; C); values from non-CF subjects are shown in grey bars (Normal); mean±SD; n=4, *=p<0.01 vs. vehicle or Trikafta®-like. (D) RNA analysis post-Ussing chamber studies: RT-PCR gel (top) and gel band quantification (bottom); the lower band is the correctly spliced CFTR (wild type, WT); n=3-4, *=p<0.01 vs. vehicle. (E) PPMO and OEC dose responses in patient-derived CF patient-derived cells. PPMO3849 and OEC were co-administered basolaterally for 6 hours at the concentrations shown in the figures; a mismatched (MM) PPMO was used as a control at 0.2 μM (left) and 1 μM (right); n=4; *=p<0.05 vs no OEC (left) and *=p<0.02 vs. vehicle (right). CFTR mRNA correction was effective in response to one-time PPMO3849 treatment.

FIGS. 4A-4C are a series of confocal microscopy images showing the correction of a splicing mutation with in vivo administration of SSO/PPMO654 in the EGFP654 mouse. EGFP654 mice received PPMO654 at 12 mg/kg IV on three successive days and thereafter one cohort received OEC (UNC7938) IV at 15 mg/kg once (n=3-4 per group). After 48 hours mice were euthanized, tissues recovered and analyzed by immunostaining with antibodies to EGFP and confocal microscopy. (A) Images of EGFP immunostaining (dashed line) and nuclear staining (*, propidium iodide) of fixed lung sections from mice treated with PPMO plus OEC, PPMO only, or vehicle. In PPMO plus OEC-treated mice, EGFP expression levels were elevated in the cells of the surface epithelium (Epi) and in the alveolar cells (Alv) compared to PPMO controls. (B) High magnification, low intensity image from PPMO plus OEC cohort to reveal EGFP expression patterns in cells displaying the morphology of secretory cells (SC). (C) Lung sections were co-stained for EGFP and for cilia with tubulin antibodies (*) to identify ciliated cells (CC) as well as cells displaying the morphology of secretory cells (SC) and basal cells (BC).

FIGS. 5A-5B show the in vivo intra-pulmonary administration of SSO/PPMO654 alone (no OEC) in the EGFP654 mouse. (A) Live images of EGFP expression (dashed line) and cilia staining (solid line) in PPMO654-corrected tracheal cell cultures derived from the EGFP654 mouse. (B) Intra-pulmonary PPMO654 efficiently corrected a splicing defect in the lung in a dose dependent manner and for at least 11 weeks after a single administration; n=3.

FIG. 6 shows the correction of the CFTR c. 1679+1643G>T splicing mutation in nasal cells from a CF patient when treated one time for 12 hours with oligonucleotides of the present invention. Cells were treated either with a control vehicle, an oligonucleotide consisting of SEQ ID NO: 1 and conjugated to the peptide consisting of SEQ ID NO: 14 as a PPMO (P7-PMO), or an oligonucleotide consisting of SEQ ID NO:9 having 2′-O-methoxyethyl (MOE) and phosphorothioate (PS) chemistry modifications (MOE+OEC). The upper band indicates the mis-spliced mRNA and the lower band indicates the wild-type (WT) mRNA; GAPDH expression was utilized as loading/experimental control.

FIGS. 7A-7B show the correction of the CFTR c. 1679+1643G>T splicing mutation in nasal cells from a CF patient when treated one time for 12 hours with oligonucleotides of the present invention. Cells were treated either with a control vehicle (n=3) or an oligonucleotide consisting of SEQ ID NO: 1 at a 1 μM concentration and incubated overnight. (A) The cells were tested 72 hours after treatment in Ussing chambers for CFTR channel activity after treatment with amiloride (Amil), followed by forskolin (FSK), VX-770 (a highly effective modulator therapy [HEMT]), and finally the CFTR inhibitor 172 (1172). The inset bar graph shows the peak of CFTR channel activity after FSK treatment. (B) After the Ussing chambers, the cells were lysed and the mRNA splicing correction was analyzed by RT-PCR (n=3). The upper band is the mis-spliced mRNA and the lower band is the WT CFTR mRNA.

FIGS. 8A-8B show the correction of the CFTR W1282X nonsense mutation in bronchial epithelial cells from a CF when treated one time for 12 hours with oligonucleotides that promote exon skipping of exon 23. The oligonucleotides used in this experiment were previously published by Michaels et al. (doi.org/10.1073/pnas.2114886119) and were conjugated with PPMO chemistry and incubated in the cell culture medium (1 μM each; overnight). The CFTR HEMT combination “ETI” (elexacaftor/VX-445 3 μM, tezacaftor/VX-661 10 μM, and ivacaftor/VX-770 1 μM) was used to increase CFTR protein activity. (A) The cells were tested 72-96 hours after oligonucleotide treatment in Ussing chambers for CFTR channel activity after treatment with Amil, followed by FSK, VX-770, and finally 1172; UTP was added as an experimental quality control. Control cultures were treated with respective vehicles. The inset bar graph shows the peak of CFTR channel activity after FSK treatment. (B) After the Ussing chambers, the cells were lysed and the mRNA splicing of exon 23 was analyzed by RT-PCR (n=3).

### DETAILED DESCRIPTION OF THE INVENTION

The present invention relates to novel SSOs to directly correct mutations, specifically, one or more mutations selected from a 1811+1.6 kb G>T (c.1679+1643G>T), 1811+1.6 kb A>G, (c. 1679+1634A>G), and/or 1811+1.6 kb T>A (c.1679+1650T>A) splicing mutation and/or a W1282X (c.3846G>A) nonsense mutation in CFTR, thereby treating cystic fibrosis (CF). Additionally, the invention may have potential therapeutic implications in various pulmonary and non-pulmonary diseases that involve splicing defects or nonsense mutations.

The present invention will now be described with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety. In case of a conflict in terminology, the present specification is controlling.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR § 1.822 and established usage. See, e.g., Patent In User Manual, 99-102 (November 1990) (U.S. Patent and Trademark Office).

Except as otherwise indicated, standard methods known to those skilled in the art may be used for cloning genes, amplifying and detecting nucleic acids, and the like. Such techniques are known to those skilled in the art. See, e.g., Green et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

### Definitions

The following terms are used in the description herein and the appended claims.

The singular forms “a” and “an” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide sequence of this invention, means a polynucleotide that consists of both the recited sequence (e.g., SEQ ID NO) and a total of ten or less (e.g., 1, 2, 3, 4, 5, 6, 7, 8, 9, or 10) additional nucleotides on the 5′ and/or 3′ ends of the recited sequence such that the function of the polynucleotide is not materially altered. The total of ten or less additional nucleotides includes the total number of additional nucleotides on both ends added together. The term “materially altered,” as applied to polynucleotides of the invention, refers to an increase or decrease in ability to correct splicing of a target mRNA by at least about 50% or more as compared to the correction achieved with a polynucleotide consisting of the recited sequence.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “wild-type protein” or “wild-type gene” or “wild-type” or grammatical variations thereof as used herein refers to a protein, gene, or organism as it is typically found in its natural, non-mutated, diseased, or otherwise changed form.

The term “partially functional” or “partially functional protein” or grammatical variations thereof as used herein refers to a protein that has at least 10% of at least one biological activity, e.g., at least 20%, 30%, 40%, 50%, 60%, or 70% or more, when compared to the wild-type version of the partially functional protein.

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject (e.g., in the case of CF, improvement of lung disease phenotypes with reduction of exacerbations and hospitalizations, and the reduction in the probability of needing a life-saving lung transplant, reduction in nasal phenotypes and sinusitis, reduction of gastrointestinal phenotypes, improvement in calorie intake and body mass, improvement in growth parameters (pediatric patients), improvement in liver and kidney phenotypes, and improvement in gynecological parameters, potential improvement in pancreatic function with early therapeutic intervention, improvement in clinical test outcomes: lung function parameters, nasal PD, and salt concentration in sweat). Those skilled in the art will appreciate that the therapeutic effects need not be complete or curative, as long as some benefit is provided to the subject.

By the terms “treat,” “treating,” or “treatment of,” it is intended that the severity of the subject's condition is reduced or at least partially improved or modified and that some alleviation, mitigation or decrease in at least one clinical symptom is achieved.

“Prevent” or “preventing” or “prevention” refer to prevention or delay of the onset of the disorder and/or a decrease in the severity of the disorder in a subject relative to the severity that would develop in the absence of the methods of the invention. The prevention can be complete, e.g., the total absence of CF in a subject. The prevention can also be partial, such that the occurrence or severity of CF in a subject is less than that which would have occurred without the present invention.

As used herein, the terms “protein” and “polypeptide” are used interchangeably and encompass both peptides and proteins, unless indicated otherwise.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention. When an oligonucleotide is produced synthetically, less common bases, such as inosine, 5-methylcytosine, 6-methyladenine, hypoxanthine and others can also be used. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The splice switching oligonucleotide or chemically-modified splice switching oligonucleotide may be constructed using chemical synthesis and enzymatic ligation reactions by procedures known in the art. For example, a splice switching oligonucleotide or chemically-modified splice switching oligonucleotide may be chemically synthesized using naturally occurring nucleotides or various modified nucleotides designed to increase the biological stability of the molecules or to increase the physical stability of the duplex formed between the splice switching oligonucleotide or chemically-modified splice switching oligonucleotide and target nucleotide sequences, e.g., phosphorothioate derivatives and acridine substituted nucleotides can be used. Examples of modified nucleotides which can be used to generate the splice switching oligonucleotide or chemically-modified splice switching oligonucleotide include, but are not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the oligonucleotide can be produced using an expression vector into which a nucleic acid encoding the splice switching oligonucleotide has been cloned.

The splice switching oligonucleotide or chemically-modified splice switching oligonucleotide can further include nucleotide sequences wherein at least one, or all, of the internucleotide bridging phosphate residues are modified phosphates, such as methyl phosphonates, methyl phosphonothioates, phosphoromorpholidates, phosphoropiperazidates, phosphoramidates, and phosphorodiamidates, including wherein one or more nucleotides form a morpholino backbone. For example, every one or every other one of the internucleotide bridging phosphate residues can be modified as described. In another non-limiting example, the splice switching oligonucleotide or chemically-modified splice switching oligonucleotide is a nucleotide sequence in which at least one, or all, of the nucleotides are modified, e.g., to contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl). In another example, one or more of the nucleotides may be a 2′-fluoro nucleotide, a 2′-O-methyl nucleotide, MOE, or a locked nucleic acid nucleotide. Other examples include 5′ constrained ethyl oligonucleotides and tricyclo-DNA oligonucleotides. For example, every one or every other one of the nucleotides can be modified as described. See also, Furdon et al., Nucleic Acids Res. 17:9193 (1989); Agrawal et al., Proc. Natl. Acad. Sci. USA 87:1401 (1990); Baker et al., Nucleic Acids Res. 18:3537 (1990); Sproat et al., Nucleic Acids Res. 17:3373 (1989); Walder and Walder, Proc. Natl. Acad. Sci. USA 85:5011 (1988); incorporated by reference herein in their entireties for their teaching of methods of making polynucleotide molecules, including those containing modified nucleotide bases).

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence. An isolated polynucleotide that includes a gene is not a fragment of a chromosome that includes such gene, but rather includes the coding region and regulatory regions associated with the gene, but no additional genes naturally found on the chromosome.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

The term “fragment,” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

By the term “express” or “expression” of a polynucleotide coding sequence, it is meant that the sequence is transcribed, and optionally, translated.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, “complementary” polynucleotides are those that are capable of base pairing according to the standard Watson-Crick complementarity rules. Specifically, purines will base pair with pyrimidines to form a combination of guanine paired with cytosine (G: C) and adenine paired with either thymine (A: T) in the case of DNA, or adenine paired with uracil (A: U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “substantially identical” or “corresponding to” means that two nucleic acid sequences have at least 60%, 70%, 80% or 90% sequence identity. In some embodiments, the two nucleic acid sequences can have at least 85%, 90%, 95%, 96%, 97%, 98%, 99% or 100% of sequence identity.

An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo, H., and Lipton, D., (Applied Math 48: 1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

### Splice Switching Oligonucleotides

The invention presents an alternative therapeutic approach by targeting CFTR at the transcriptional level. The invention consists of splice switching oligonucleotides that can complementarily bind CFTR pre-mRNA and modulate the activity of the spliceosome, inducing correction of one or more of the mutations listed in Table 1 and production of either full length wild-type CFTR mRNA or CFTR mRNA that encodes for a partially functional CFTR protein. Traditionally, oligonucleotides have faced clinical limitations due to their lack of tissue specificity, rapid degradation within the body, and immune activation. However, the synthetic SSOs herein contain novel chemical modifications that confer drug-like properties, which will protect them from in vivo degradation.

TABLE 1
CFTR mutations that cause CF

| Mutation legacy name | Mutation new name | Mutation type |
|---|---|---|
| 1811 + 1.6 kb G > T | c.1679 + 1643G > T | Splicing |
| 1811 + 1.6 kb A > G | c.1679 + 1634A > G | Splicing |
| 1811 + 1.6 kb T > A | c.1679 + 1650T > A | Splicing |
| W1282X | c.3846G > A | Nonsense |

Accordingly, one aspect of the invention relates to a splice switching oligonucleotide, wherein the nucleotide sequence is at least 80% complementary (e.g., at least 85%, 90%, 91%, 92%, 93%, 94%, 95%, 96%, 97%, 98%, or 99% complementary) to either an intronic or exonic region of the CFTR gene, the region consisting essentially of about 15 to about 40 consecutive nucleotides, e.g., about 20 to about 35 consecutive nucleotides, about 20 to about 30 consecutive nucleotides, or any range therein; wherein the splice switching oligonucleotide corrects an intronic splicing mutation or exonic nonsense mutation in the CFTR gene. The splice switching oligonucleotide provides increased expression of either a wild-type or partially functional CFTR protein in a cell as compared to cells without the splice switching oligonucleotide (e.g., a control cell, non-transfected cell, or a cell expressing mutant CFTR or minimal or no CFTR). In some embodiments, expression of wild-type CFTR or partially functional CFTR is at least about 5% compared to a normal cell (e.g., a cell without a CFTR mutation), e.g., at least about 5%, 10%, 20%, 30%, 40%, 50%, 60%, 70%, 80%, 90%, 95%, or more. The sequence of the human CFTR gene on which the oligonucleotide is based is known in the art and can be found, e.g., in Accession No. NM_000492, incorporated herein by reference in its entirety. The CFTR mutations 1811+1.6 kb G>T (c.1679+1643G>T), 1811+1.6 kb A>G (c.1679+1634A>G), 1811+1.6 kb T>A (c.1679+1650T>A), and W1282X (c.3846G>A) are depicted in Accession Nos. rs397508261, rs397508266, and rs77010898, respectively, in the SNP database (dbSNP), incorporated herein by reference in their entirety.

Additional nucleotides can be added at the 3′ end, the 5′ end, or both the 3′ and 5′ ends to facilitate manipulation of the splice switching oligonucleotide but that do not materially affect the basic characteristics or function of the splice switching oligonucleotide. Additionally, one or two nucleotides can be deleted from one or both ends of any of the sequences disclosed herein that do not materially affect the basic characteristics or function of the splice switching oligonucleotide. The term “materially affect” as used herein refers to a change in the ability to correct expression of the wild-type protein encoded by the mRNA by more than about 50%, e.g., more than about 50%, 55%, 60%, 65%, 70%, 75%, 80%, 90%, 100%, or more.

In particular embodiments, the present invention provides a splice switching oligonucleotide containing a nucleotide sequence that is fully complementary to a region of the target gene. However, it is to be understood that 100% complementarity between the splice switching oligonucleotide and the target sequence is not required to practice the present invention. Thus, sequence variations that might be expected due to genetic mutation, strain polymorphism, or evolutionary divergence can be tolerated. Oligonucleotides with insertions, deletions, and single point mutations relative to the target sequence may also be effective for inhibition.

In some embodiments, the nucleotide sequence of the splice switching oligonucleotide comprises at least 5 consecutive nucleotides of the sequence of any one of SEQ ID NOS: 1-13 (e.g., at least 5, 6, 7, 8, 9, 10, 11, 12, 13, 14, 15, 16, 17, 18, 19, 20, or more consecutive nucleotides of the sequence of any one of SEQ ID NOS: 1-13). In some embodiments, the splice switching oligonucleotide is about 15 to about 30 nucleotides in length, e.g., about 20 to about 25 nucleotides in length (e.g., about 15, 16, 17, 18, 19, 20, 21, 22, 23, 24, 25, 26, 27, 28, 29, or about 30 nucleotides in length or any range therein). In some embodiments, the splice switching oligonucleotide is less than about 30 nucleotides in length, e.g., less than about 29, 28, 27, 26, 25, 24, 23, 22, 21, or 20 nucleotides in length.

In some embodiments, the nucleotide sequence of the splice switching oligonucleotide comprises a nucleotide sequence that is at least about 70% identical to the nucleotide sequence of any of SEQ ID NOS: 1-13, e.g., at least about 70%, 75%, 80%, 85%, 90%, 91%, 92%, 93%, 94%, 95%, 96%, 97%, 98%, 99%, or more identical to the nucleotide sequence of any of SEQ ID NOS: 1-13. In some embodiments, the nucleotide sequence of the splice switching oligonucleotide comprises, consists essentially of, or consist of the nucleotide sequence of any of SEQ ID NOS: 1-13.

TABLE 2
Oligonucleotide sequences

| Sequence | Oligonucleotide | |
|---|---|---|
| number | sequence | CFTR mutation target |
| SEQ ID | CAGGTGTGATTGATA | 1811 + 1.6 kb G > T |
| NO: 1 | GTAAACTTAC | 1811 + 1.6 kb A > G |
| | | 1811 + 1.6 kb T > A |
| SEQ ID | GATTGATAGTAAACT | 1811 + 1.6 kb G > T |
| NO: 2 | TACTTATATC | 1811 + 1.6 kb A > G |
| | | 1811 + 1.6 kb T > A |
| SEQ ID | GTAAACTTACTTATA | 1811 + 1.6 kb G > T |
| NO: 3 | TCTCAAGTAC | 1811 + 1.6 kb A > G |
| | | 1811 + 1.6 kb T > A |
| SEQ ID | CAGGTGTGATTGATA | 1811 + 1.6 kb G > T |
| NO: 4 | GTAACCTTAC | 1811 + 1.6 kb A > G |
| | | 1811 + 1.6 kb T > A |
| SEQ ID | GTAACCTTACTTACA | 1811 + 1.6 kb A > G |
| NO: 5 | TCTCAAGTAC | 1811 + 1.6 kb G > T |
| | | 1811 + 1.6 kb T > A |
| SEQ ID | GTAACCTTACTTATA | 1811 + 1.6 kb A > G |
| NO: 6 | TCTCAAGTAC | 1811 + 1.6 kb G > T |
| | | 1811 + 1.6 kb T > A |
| SEQ ID | TATCTCAAGTACATA | 1811 + 1.6 kb A > G |
| NO: 7 | GGATTCTCTG | 1811 + 1.6 kb G > T |
| | | 1811 + 1.6 kb T > A |
| SEQ ID | GATAGTAACCTTACT | 1811 + 1.6 kb A > G |
| NO: 8 | TATATCTCAA | 1811 + 1.6 kb G > T |
| | | 1811 + 1.6 kb T > A |
| SEQ ID | CAGGTGTGATTGATA | 1811 + 1.6 kb G > T |
| NO: 9 | GTAAAC | 1811 + 1.6 kb A > G |
| | | 1811 + 1.6 kb T > A |
| SEQ ID | CATGTAATTGTTGGT | 1811 + 1.6 kb G > T |
| NO: 10 | ACTTCTGTAA | 1811 + 1.6 kb A > G |
| | | 1811 + 1.6 kb T > A |
| SEQ ID | TCTTCCCAAGAGGCC | W1282X |
| NO: 11 | CACCTATAAG | |
| SEQ ID | CACCTGTGGTATCAC | W1282X |
| NO: 12 | TCCAAAGGCT | |
| SEQ ID | GGCTAAGTCCTTTTG | W1282X |
| NO: 13 | CTCACCTGTG | |

In particular embodiments, the present invention provides a splice switching oligonucleotide containing a nucleotide sequence that is fully complementary to an intronic or exonic region of the target gene for correcting a splicing mutation. However, it is to be understood that the splice switching oligonucleotide does not necessarily need to overlap the intronic or exonic region containing the mutation. In some embodiments, the splice switching oligonucleotide is complementary to a region of the gene up to 100 nucleotides (e.g., up to 100 nucleotides, 75 nucleotides, 50 nucleotides, 25 nucleotides, 20 nucleotides, 15 nucleotides, 10 nucleotides, or 5 nucleotides) upstream of the mutation. In some embodiments, the splice switching oligonucleotide is complementary to a region of the gene up to 100 nucleotides (e.g., up to 100 nucleotides, 75 nucleotides, 50 nucleotides, 25 nucleotides, 20 nucleotides, 15 nucleotides, 10 nucleotides, or 5 nucleotides) downstream of the mutation. Thus, complementary sequences do not necessarily need to overlap the mutation in order to practice the invention. A splice switching oligonucleotide not overlapping the mutation may also be effective for correcting the splicing mutation and expressing normal CFTR.

In some embodiments, nuclear localization signals can be used to enhance the targeting of the splice switching oligonucleotide into the proximity of the nucleus and/or its entry into the nucleus. Such nuclear localization signals can be a protein or a peptide such as the SV40 large Tag NLS or the nucleoplasmin NLS. These nuclear localization signals interact with a variety of nuclear transport factors such as the NLS receptor (karyopherin alpha) which then interacts with karyopherin beta.

In some embodiments, the splice switching oligonucleotide is conjugated (e.g., through a morpholino group) to a peptide that enhances the delivery and/or activity of the oligonucleotide, e.g., a cationic peptide. In one embodiment, the peptide is at least 70% identical to RXRRXRRXRRXRXB (SEQ ID NO:14), e.g., at least 70%, 75%, 80%, 85%, 90%, 91%, 92%, 93%, 94%, 95%, 96%, 97%, 98%, 99%, or 100% identical to RXRRXRRXRRXRXB (SEQ ID NO: 14), wherein R is arginine, B is β-alanine, and X is 6-aminohexanoic acid. In some embodiments, the peptide is a short cationic peptide (e.g., about 5 6, 7, 8, 9, 10, 11, 12, or about 13 amino acids in length). In some embodiments, the short cationic peptide provides an improved therapeutic index to the splice switching oligonucleotide. In some embodiments, the peptide is a cyclic peptide. In some embodiments, the peptide is hydrophobic (e.g., the peptide sequence comprises a majority of hydrophobic amino acid residues). In some embodiments, the peptide is hydrophilic (e.g., the peptide sequence comprises a majority of hydrophilic amino acid residues).

In some embodiments, one or more nucleotides of the splice switching oligonucleotide are chemically modified nucleotides and/or the backbone of the oligonucleotide is chemically modified. In some embodiments, one, all, or fewer than all the nucleotides are modified. In some embodiments, at least 1, 2, 3, 4, 5, 6, 7, 8, 9, 10, 11, 12, 13, 14, 15, 16, 17, 18, 19, or 20 or more nucleotides are modified. In some embodiments, the splice switching oligonucleotide has more than one type of modification. In some embodiments, the splice switching oligonucleotide comprises one or more phosphorodiamidate morpholino nucleotides, forming at least part of a morpholino backbone. In other embodiments, all the nucleotides of the splice switching oligonucleotide are phosphorodiamidate morpholino nucleotides, forming a morpholino backbone.

In some embodiments, the splice switching oligonucleotide comprises one or more MOE nucleotides. In other embodiments, all the nucleotides of the splice switching oligonucleotide are 2′-O-methoxyethyl nucleotides. The MOE-modified oligonucleotide may have additional modifications, e.g., a phosphorothioate backbone.

Delivery into Target Cells and/or Nucleus

As used herein, the terms “contacting,” “introducing” and “administering” are used interchangeably, and refer to a process by which SSOs of the present invention or a nucleic acid molecule encoding a SSO of this invention is delivered to a cell, in order to inhibit or alter or modify expression of a target gene. The SSO may be administered in a number of ways, including, but not limited to, direct introduction into a cell (i.e., intracellularly) and/or extracellular introduction into a cavity, interstitial space, or into the circulation of the organism, e.g., the nose, lung, ear, eye, or intestine.

“Introducing” in the context of a cell or organism means presenting the nucleic acid molecule to the organism and/or cell in such a manner that the nucleic acid molecule gains access to the interior of a cell. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into cells in a single transfection event or in separate transfection events. Thus, the term “transfection” as used herein refers to the introduction of a heterologous nucleic acid into a cell. Transfection of a cell may be stable or transient.

“Transient transfection” in the context of a polynucleotide means that a polynucleotide is introduced into the cell and does not integrate into the genome of the cell.

According to the present invention, a splice switching oligonucleotide as described herein may be delivered as naked nucleic acid (unpackaged) or via delivery vehicles. As used herein, the terms “delivery vehicle,” “transfer vehicle,” “nanoparticle” or grammatical equivalent, are used interchangeably.

In some embodiments, a splice switching oligonucleotide may be delivered via a single delivery vehicle. In some embodiments, a splice switching oligonucleotide may be delivered via one or more delivery vehicles each of a different composition. According to various embodiments, suitable delivery vehicles include, but are not limited to polymer based carriers, such as polyethyleneimine (PEI), lipid nanoparticles and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic polyconjugates), dry powder formulations, plasmids, viruses, calcium phosphate nucleotides, aptamers, peptides and other vectorial tags, and vehicles suitable for e.g., intravenous (e.g., systemic or organ localized vessels), subcutaneous, intraperitoneal, intramuscular, intranasal, intrapulmonary, intra-ductal, ocular, otic, or intrathecal delivery, direct injection or instillation in an organ, or use in conjunction with methods that apply electrical fields to improve drug delivery into tissues (e.g., electroporation, iontophoresis).

In some embodiments, a polynucleotide of this invention can be delivered to a cell in vivo by lipofection. Synthetic cationic lipids designed to limit the difficulties and dangers encountered with liposome-mediated transfection can be used to prepare liposomes for in vivo transfection of a nucleotide sequence of this invention (Felgner et al., Proc. Natl. Acad. Sci. USA 84:7413 (1987); Mackey, et al., Proc. Natl. Acad. Sci. U.S.A. 85:8027 (1988); and Ulmer et al., Science 259:1745 (1993)). The use of cationic lipids may promote encapsulation of negatively charged nucleic acids, and also promote fusion with negatively charged cell membranes (Felgner et al., Science 337:387 (1989)). Particularly useful lipid compounds and compositions for transfer of nucleic acids are described in International Patent Publications WO95/18863 and WO96/17823, and in U.S. Pat. No. 5,459,127. The use of lipofection to introduce exogenous nucleotide sequences into specific organs in vivo has certain practical advantages. Molecular targeting of liposomes to specific cells represents one area of benefit. It is clear that directing transfection to particular cell types would be particularly preferred in a tissue with cellular heterogeneity, such as lung, pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules can be coupled to liposomes chemically.

In various embodiments, other molecules can be used for facilitating delivery of a nucleic acid in vivo, such as a cationic oligopeptide (e.g., WO95/21931), peptides derived from nucleic acid binding proteins (e.g., WO96/25508), and/or a cationic polymer (e.g., WO95/21931) and/or peptides as described above.

In particular embodiments, a splice switching oligonucleotide according to the present invention is administered to the subject to treat, delay the onset of and/or prevent CF.

Thus, as one aspect, the invention further encompasses a method of delivering a splice switching oligonucleotide to a subject, comprising administering to the subject an effective amount of the splice switching oligonucleotide, thereby delivering the splice switching oligonucleotide to the subject.

In another aspect, the invention further encompasses a method of treating, delaying the onset of, and/or preventing CF in a subject in need thereof, comprising administering to the subject a therapeutically effective amount of a splice switching oligonucleotide, thereby treating or delaying the onset of CF in the subject.

In the methods of the invention, the subject may be one has been diagnosed with CF having one or more 1811+1.6 kb G>T (c.1679+1643G>T), 1811+1.6 kb A>G (c.1679+1634A>G), and/or 1811+1.6 kb T>A (c.1679+1650T>A) splicing mutation and/or a W1282X (c.3846G>A) nonsense mutation or is suspected of having CF with one or more 1811+1.6 kb G>T (c. 1679+1643G>T), 1811+1.6 kb A>G (c.1679+1634A>G), and/or 1811+1.6 kb T>A (c. 1679+1650T>A) splicing mutation and/or a W1282X (c.3846G>A) nonsense mutation. Any of these mutations can be present at least in one allele in combination with the same or other CF-causing mutations on the other allele, in the genome of the somatic cells of an individual. In certain embodiments, the subject is an infant or child, e.g., less than 18 years old, e.g., less than 18, 17, 16, 15, 14, 13, 12, 11, 10, 9, 8, 7, 6, 5, 4, 3, 2, or 1 years old.

In some embodiments, an oligonucleotide endosomal compound is administered concurrently or sequentially with a splice switching oligonucleotide. The oligonucleotide endosomal compound can increase the delivery and/or increase the activity of the splice switching oligonucleotide in the cell. The oligonucleotide endosomal compound may be present in the same composition as the oligonucleotide or in a separate composition. Exemplary oligonucleotide endosomal compounds can be found in, for example, U.S. Pat. No. 10,266,823, which is herein incorporated by reference in its entirety. For further example, oligonucleotide endosomal compounds can be compounds of Formula I:

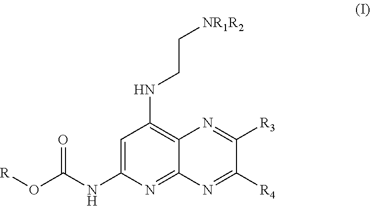

wherein:
R is ethyl or a linking group (preferably ethyl);
R₁ is methyl or a linking group (preferably methyl);
R₂ is methyl;
R₃ and R₄ are each independently H, lower alkyl; lower alkoxy, halo, amino, aryl, or heteroaryl;
or a pharmaceutically acceptable salt thereof.

In some embodiments, a CFTR modulator compound is administered concurrently or sequentially with a splice switching oligonucleotide, optionally concurrently or sequentially with a combination of a splice switching oligonucleotide and OEC. The CFTR modulator compound can increase the channel activity of partially functional CFTR proteins through one or more mechanisms (e.g., when the splice switching oligonucleotide produces a partial correction of CFTR). The CFTR modulator compound may be present in the same composition as the oligonucleotide or in a separate composition. In some embodiments, the CFTR modulator is a highly effective modulator treatment (HEMT). In some embodiments, the HEMT is elexacaftor (e.g., VX-445), tezacaftor (e.g., VX-661), ivacaftor (e.g., VX-770), or any combination thereof. Exemplary CFTR compounds include, but are not limited to, Orkambi®, Trikafta®, Kalydeco®) (e.g., ivacaftor, e.g., VX-770), and Symdeko®.

### Subjects, Pharmaceutical Formulations, and Modes of Administration

Splice switching oligonucleotides (SSOs) according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, rodents, lagomorphs, etc. Human subjects include neonates, infants, juveniles and adults.

In particular embodiments, the present invention provides a pharmaceutical composition comprising a splice switching oligonucleotide of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

One aspect of the present invention is a method of transferring a nucleic acid to a cell in vitro. The splice switching oligonucleotide may be introduced into the cells according to standard transfection methods suitable for the particular target cells. The amount of splice switching oligonucleotides to administer can vary, depending upon the target cell type and number, and the particular splice switching oligonucleotide, and can be determined by those of skill in the art without undue experimentation.

The cell(s) into which the splice switching oligonucleotide is introduced can be of any type. Moreover, the cell can be from any species of origin, as indicated above. The cell(s) can be from airway epithelia or any tissue effected by CF such as epithelial cells from lung tissue (e.g., nasal, bronchial, bronchiolar, tracheal, alveolar (e.g., alveolar type II (ATII)), muscle, sweat duct/gland, mammary, intestinal, colon, duodenum, jejunum, pancreatic, ocular (e.g., lens and/or retinal), lachrymal duct, otic epithelia, sperm, circulatory endothelium, peritoneal mesothelium, pleural cavity, pericardial cavity, esophageal epithelium, stomach, gingival, vaginal, corneal, oral, kidney tubule, ovarian, bronchial, airway gland, mammary gland, sweat gland, salivary gland, gastric, intestinal, uterine, tracheal, fallopian tube, ocular conjunctiva, urethra, pharynx, brain ventricles, bone marrow and blood, muscle, small intestine, large intestine, gall bladder, thyroid follicles, anus, vas deferens, lymph vessel, skin, endometrium, middle ear, epididymis, and/or cervix epithelia.

A further aspect of the invention is a method of administering the splice switching oligonucleotide to subjects. Administration of the splice switching oligonucleotide according to the present invention to a human subject or an animal in need thereof can be by any means known in the art. Optionally, the splice switching oligonucleotide is delivered in a treatment effective or prevention effective dose in a pharmaceutically acceptable carrier.

Dosages of the splice switching oligonucleotide to be administered to a subject depend upon the mode of administration, the disease or condition to be treated and/or prevented, the individual subject's condition, the particular splice switching oligonucleotide, and the nucleic acid to be delivered, and the like, and can be determined in a routine manner.

In particular embodiments, more than one administration (e.g., two, three, four or more administrations) may be employed to achieve the desired level of gene expression over a period of various intervals, e.g., daily, weekly, monthly, yearly, etc.

In particular embodiments, administration can be local or systemic. For example, delivery of SSOs encompasses situations in which a SSO is delivered to a target tissue and the corrected protein is expressed and retained within the target tissue (also referred to as “local distribution” or “local delivery”), and situations in which a SSO is delivered to a target tissue and the corrected protein is expressed and secreted into patient's circulation system (e.g., serum) and systematically distributed and taken up by other tissues (also referred to as “systemic distribution” or “systemic delivery). The delivery can be to airway epithelial cells or any tissue affected by CF such as epithelial cells from lung, nose, ear, eye, nervous system, and the gastrointestinal and reproductive tract tissues.

Injectables can be prepared in conventional forms, either as liquid solutions or suspensions, solid forms suitable for solution or suspension in liquid prior to injection, or as emulsions. Alternatively, one may administer the splice switching oligonucleotide of the invention in a local manner, for example, in a depot or sustained-release formulation.

Non-limiting examples of formulations of the invention include those suitable for oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal, intrathecal, intraocular, intra-ear), topical (i.e., both skin and mucosal surfaces, including airway surfaces), intranasal, transdermal, intraarticular, intracranial, intrathecal, cerebrospinal, and inhalation administration, otic administration, ocular administration, administration to the liver by intraportal delivery, as well as direct organ injection or infusion (e.g., into the liver, into the pancreas, into a limb, into the brain or spinal cord for delivery to the central nervous system, into the pancreas, or into a tumor or the tissue surrounding a tumor). The most suitable route in any given case will depend on the nature and severity of the condition being treated and on the nature of the particular compound which is being used.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

The oligonucleotide can alternatively be formulated for nasal, otic, or ocular administration or otherwise administered to the lungs of a subject by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Having described the present invention, the same will be explained in greater detail in the following examples, which are included herein for illustration purposes only, and which are not intended to be limiting to the invention.

### Example 1

Oligonucleotide (SSO) delivery to CF-relevant tissues. A major obstacle for oligonucleotide therapeutics has been their inefficient delivery to extra-hepatic organs, with lung being an elusive target. Moreover, >90% of intracellular non-viral nucleic acids are trapped in endosomes, resulting in their degradation or exocytosis. Chemical modification and combination with other molecules can significantly increase the oligonucleotide biodistribution to many organs and their intracellular distribution. This invention utilizes a novel oligonucleotide approach to increase delivery and activity, as well as to target the lung and intestine.

The primary component in this approach is the SSO, a peptide-conjugate of phosphorodiamidate morpholino oligomer (PMO). The morpholino oligomer or PMO is designed with a nucleic base sequence to specifically target a mutation in the pre-mRNA. The morpholino chemistry produces high stability, durability, good biodistribution, and low toxicity. The PMO is covalently-linked to a small peptide to increase membrane translocation and intracellular delivery; the peptide PMO conjugate is named PPMO. PPMOs have improved delivery, durability and, thus, high potency.

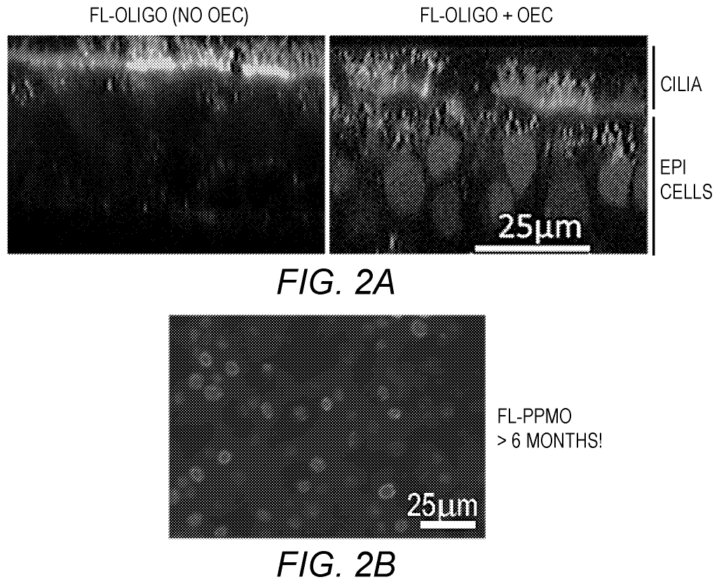

The second, and optional, component in this approach is an oligonucleotide endosomal compound (OEC). These are small molecules that enhance escape from endosomes improving the intracellular delivery of SSOs to the nuclear pre-mRNA. Although PPMOs themselves have improved intracellular delivery in vitro and may not require OEC in the mouse lung (FIGS. 2A-2B (published in Kreda, Curr. Opin. Pharmacol. 66:102271 (2022)) and 4A-4C (published in Dang et al., Nucleic Acid Res. 49:6100 (2021))), having the flexibility to use a two-component strategy with additive effects permits therapeutic approaches to reduce the effective doses of each entity for an optimal therapeutic index in the clinic.

### Example 2

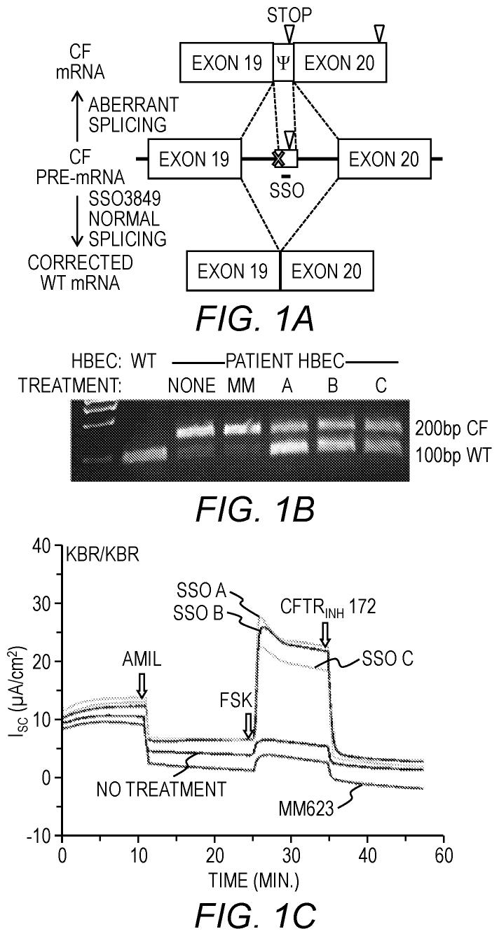

Therapeutic SSOs used to modulate CFTR splicing. The schematic in FIG. 1A depicts the effects of the 3849+10kbC>T splicing mutation and the strategy to correct it with SSOs (published in Kreda, Curr. Opin. Pharmacol. 66:102271 (2022)). FIGS. 1B and 1C show the activity of three SSOs targeted against the 3849+10kbC>T mutation and a mismatched SSO that were delivered by electroporation with 100% efficiency into patient HBECs with said splicing mutation. SSOs dramatically improved CFTR function, with activity levels within the range of experimentally-comparable non-CF airway cells (post-amiloride ΔIsc forskolin peak 20-50 μA/cm²). This SSO strategy fully corrected 3849+10kbC>T.

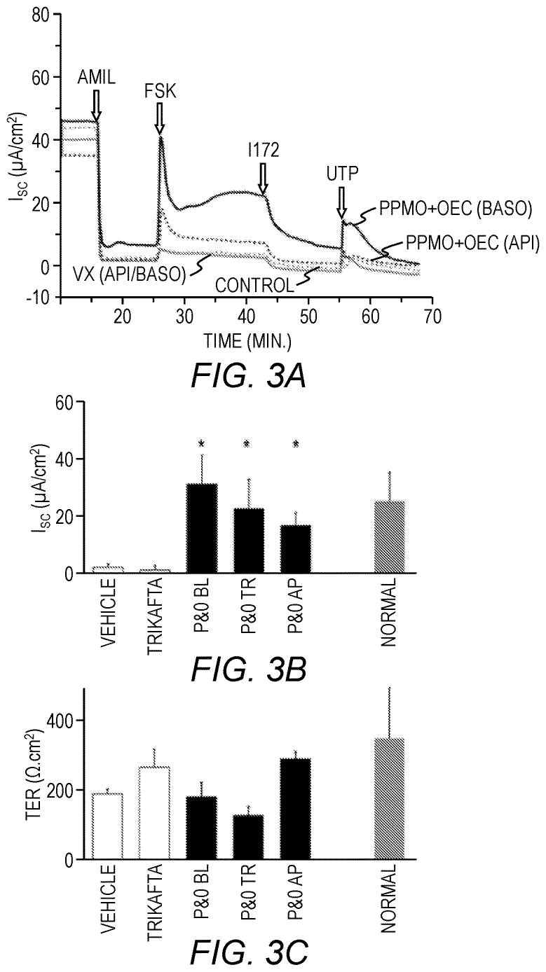

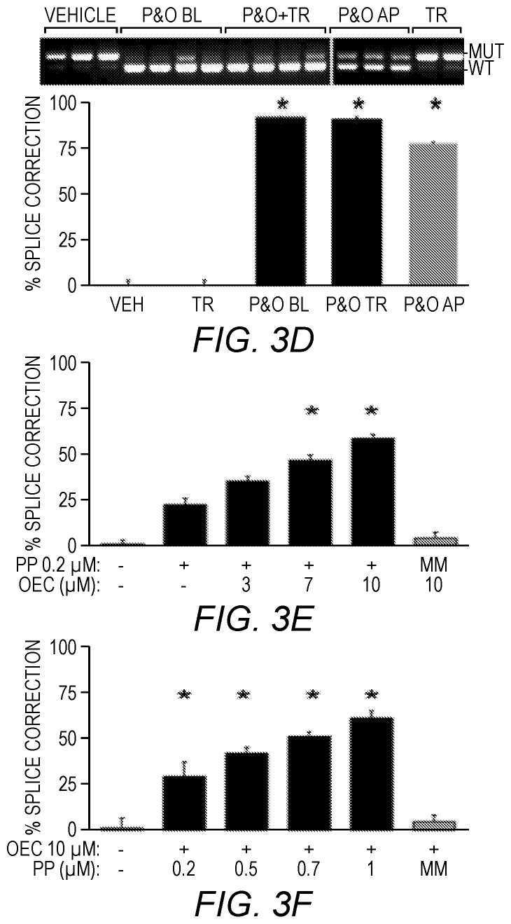

Moreover, one treatment of SSO/PPMO corrected the 3849-10kbC>T mRNA splicing mutation and restored normal levels of CFTR activity in differentiated HBEC derived from a CF patient (FIGS. 3A-3E (published in Dang et al., Nucleic Acid Res. 49:6100 (2021))). Apical or basolateral SSO administration were both efficient in fully correcting CFTR mRNA and function. Oligonucleotide treatment (PPMO+OEC) did not produce cytotoxicity as revealed by RNAseq analyses and transepithelial resistances were not significantly different in the oligonucleotide-vs. vehicle-treated groups (FIG. 3C). Importantly, this patient's HBEC did not respond to treatment with Orkambi® or Trikafta® (FIGS. 3A and 3B). Combination of Trikafta® with the SSO did not further increase CFTR activity, likely because the SSO produced full correction of CFTR mRNA and activity at the doses used in these experiments (FIGS. 3A and 3D). Dose-response studies in FIG. 3E show the activity profile of the SSO and OEC in cells derived from the CF HBEC used in FIGS. 3A and 3D.

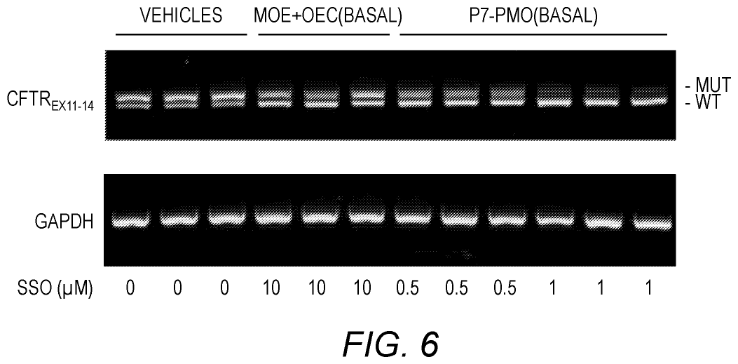

The oligonucleotides consisting of SEQ ID NO: 1 and SEQ ID NO:9 were tested in CF patient nasal cells to correct the 1811+1.6 kb G>T mutation. The former consisted of PMO base pair linkages and conjugated at the 3′ end to the peptide sequence consisting of SEQ ID NO: 14 (e.g., PPMO chemistry), while the latter consisted of PS base pair linkages and MOE modifications on the base pairs (e.g., MOE/PS chemistry). Cells were plated on permeable supports and differentiated for three weeks according to Dang et al 2021 (doi.org/10.1093/nar/gkab488). Cells were treated either with a control vehicle or the previously mentioned oligonucleotide. The oligonucleotide treatments were added to the medium only once, for 12 hours at the concentrations shown (FIG. 6). Cells treated with the oligonucleotide consisting of SEQ ID NO:9 also received OEC UNC7938 (10 μM, 1 hour). Cells were lysed 72 hours after oligonucleotide treatment and the mRNA splicing correction was analyzed by RT-PCR. The MOE/PS chemistry oligonucleotide elicited modest splicing correction in CFTR, however the PPMO chemistry oligonucleotide elicited significant splicing correction in a dose dependent manner, and full correction of the splicing was seen at the 1 μM dose (FIG. 6).

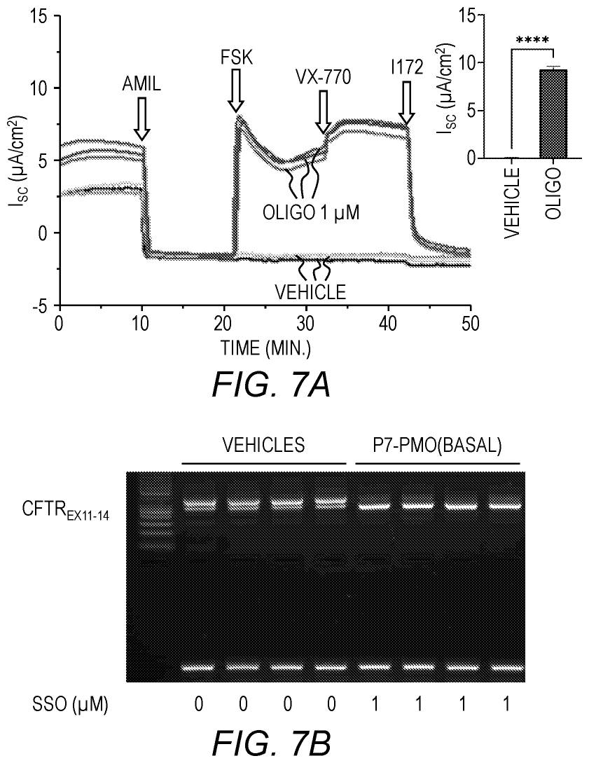

To confirm that these treatments restored normal CFTR channel activity, CF patient nasal cells with the 1811+1.6 kb G>T mutation were treated with the oligonucleotide consisting of SEQ ID NO: 1 with the PPMO chemistry using the peptide sequence consisting of SEQ ID NO:14 and then analyzed via Ussing chambers (FIG. 7A). Cells were plated on permeable supports and differentiated for three weeks according to Dang et al 2021 (doi.org/10.1093/nar/gkab488). Cells were treated either with a control vehicle (n=3) or an oligonucleotide consisting of SEQ ID NO: 1 at a 1 μM concentration and incubated overnight. The cells were tested 72 hours after treatment in Ussing chambers for CFTR channel activity; amiloride (Amil) was added first to block sodium transport, followed by forskolin (FSK) to stimulate CFTR activity, VX-770 was then added as a highly effective modulator therapy (HEMT) to increase channel activity, and lastly the CFTR inhibitor 172 (1172) was added to confirm CFTR activity. These cells were further lysed in order to examine the splicing correction at the mRNA level via RT-PCR (FIG. 7B). The data indicates that the oligonucleotide treatment fully restored CFTR channel activity.

Without wishing to be bound by any particular theory, the proximity of the 1811+1.6 kb CFTR mutations (e.g., 1811+1.6 kb G>T, 1811+1.6 kb A>G, and 1811+1.6 kb T>A) indicates that a single oligonucleotide sequence could be used to alter the splicing activity in the CFTR gene if any one of these mutations is present and thus treat multiple different patient populations.

### Example 3

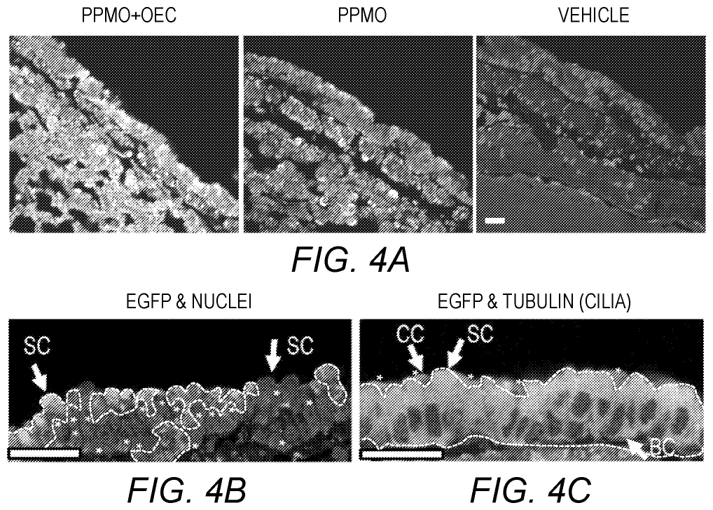

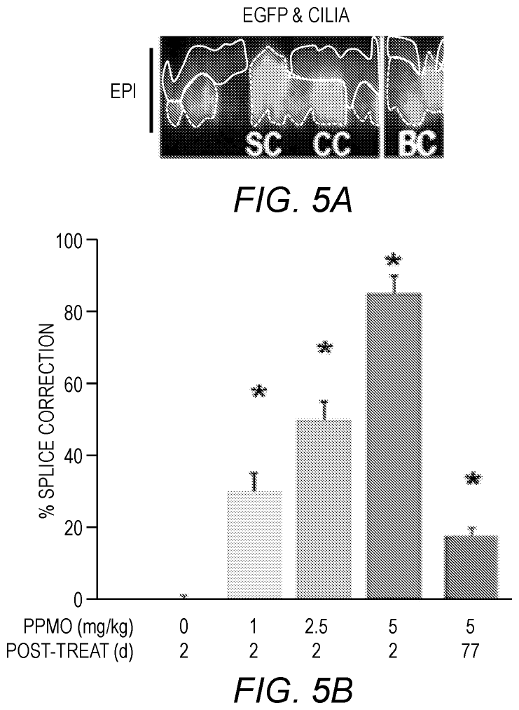

Oligonucleotide (SSO) delivery to mice in vivo. In vivo studies were performed in the EGFP654 mouse, a reporter mouse expressing an inactive EGFP due to a beta-globin splicing mutation. One low dose (5-12 mg/kg) intravenous treatment of the specific SSO PPMO654 (+OEC) elicited significant splicing correction in the lung epithelia (FIGS. 4A-4C), including in ciliated (CC), secretory (SC) and basal/stem-like (BC) epithelial cells (FIGS. 4A-4C and 5A). The corrective activity of the PPMO in vivo was observed in the lung and intestine for at least 3 weeks. Data show that intra-pulmonary delivery of PPMO (1-5 mg/kg, intra-tracheal; with no OEC) exhibited significant splicing correction activity in the lung and trachea in a dose-dependent manner and for at last 11 weeks (77 days) after a single administration (FIG. 5B). No toxicity was observed in these studies.

### Example 4

Therapeutic SSOs used to correct CFTR nonsense mutations. Nonsense mutations, such as W1282X in the CFTR gene, may be corrected by oligonucleotides that promote the skipping of the exon containing the mutation, thus bypassing the mutated site entirely. The stabilized mRNA will often translate only a partially functional protein, however these can often be corrected with further treatment of therapeutics that target this protein. In CFTR, protein modulators such as HEMTs can be used in combination with the splicing oligonucleotides of the present invention to correct the nonsense mutations such as W1282X. Correction of these types of mutations is critical, as they are often very difficult to target and produce a near 100% loss of function from the affected gene.

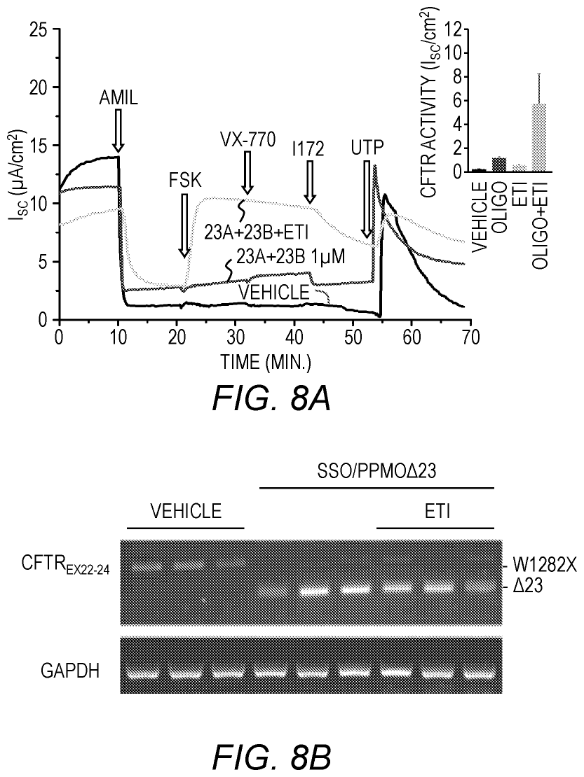

To test this, bronchial epithelial cells from a CF patient were treated with oligonucleotides that promote exon skipping of exon 23 in the CFTR gene. Cells were plated on permeable supports and differentiated for three weeks according to Dang et al 2021 (doi.org/10.1093/nar/gkab488). The oligonucleotides tested were previously published by Michaels et al. (doi.org/10.1073/pnas.2114886119) and were conjugated with PPMO chemistry using the peptide sequence consisting of SEQ ID NO:14 and incubated in the cell culture medium (1 μM each; overnight). The following day, the CFTR HEMT combination “ETI” (elexacaftor/VX-445 3 μM, tezacaftor/VX-661 10 μM, and ivacaftor/VX-770 1 μM) was added and the cells were incubated for 48 hours. The cells were tested 72 hours after oligonucleotide treatment in Ussing chambers for CFTR channel activity (FIG. 8B). As before, amil, FSK, VX-770, and finally 1172 was added to interrogate channel activity; UTP was added as an experimental quality control. Control cultures were treated with respective vehicles. After the Ussing chambers, the cells were lysed and the mRNA splicing correction was analyzed by RT-PCR (n=3) (FIG. 8A). The data demonstrates that the oligonucleotides elicited ˜100% exon 23 skipping by RT-PCR and that the CFTR channel activity was significantly corrected with further enhancement by the CFTR modulators. No medium enhancers or delivery molecules/maneuvers were utilized. These data, when compared to the previously published data, showed that a shorter treatment at a significantly reduced dose was over 80 times more efficient compared to the unconjugated oligos. Several novel oligonucleotide sequences (SEQ ID NOS: 11-13) were generated by our lab which are predicted to have similar efficacies in promoting exon 23 skipping as those shown here.

All publications, patents, and patent applications are herein incorporated by reference to the same extent as if each individual publication, patent, or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

1. A splice switching oligonucleotide for correcting one or more of a 1811+1.6 kb G>T (c.1679+1643G>T), 1811+1.6 kb A>G (c.1679+1634A>G), 1811+1.6 kb T>A (c. 1679+1650T>A), or W1282X (c.3846G>A) mutation in a human CFTR gene, wherein the oligonucleotide specifically hybridizes to an mRNA produced from the mutated CFTR gene at a site within 100 nucleotides of the mutation.

2. The splice switching oligonucleotide of claim 1, which specifically hybridizes to an mRNA produced from the mutated CFTR gene at a site within 25 nucleotides of the mutation.

3. The splice switching oligonucleotide of claim 1, comprising at least 5 consecutive nucleotides of the sequence of any one of SEQ ID NOS: 1-13.

4. The splice switching oligonucleotide of claim 1, wherein the oligonucleotide comprises a sequence at least 70% identical to any one of SEQ ID NOS: 1-13.

5. The splice switching oligonucleotide of claim 4, comprising the nucleotide sequence of any one of SEQ ID NOS: 1-13.

6-17. (canceled)

18. The splice switching oligonucleotide of claim 1, wherein one or more nucleotides are chemically modified.

19. The splice switching oligonucleotide of claim 18, wherein all of the nucleotides are chemically modified.

20. The splice switching oligonucleotide of claim 18, wherein the oligonucleotide comprises a phosphorodiamidate morpholino backbone.

21. The splice switching oligonucleotide of claim 18, wherein the oligonucleotide comprises one or more 2′-O-methoxyethyl or 5′ constrained ethyl nucleotides.

22. The splice switching oligonucleotide of claim 1, further comprising a peptide conjugated to the oligonucleotide.

23. The splice switching oligonucleotide of claim 22, wherein the peptide is a cationic peptide.

24. The splice switching oligonucleotide of claim 22, wherein the peptide comprises a sequence at least 90% identical to RXRRXRRXRRXRXB (SEQ ID NO: 14), wherein R is arginine, B is β-alanine, and X is 6-aminohexanoic acid.

25. The splice switching oligonucleotide of claim 24, comprising the peptide sequence of RXRRXRRXRRXRXB (SEQ ID NO:14), wherein R is arginine, B is β-alanine, and X is 6-aminohexanoic acid.

26. A pharmaceutical composition comprising one or more splice switching oligonucleotide of claim 1 in a pharmaceutically acceptable carrier.

27. The pharmaceutical composition of claim 26, further comprising an oligonucleotide endosomal compound.

28. A method of correcting one or more of a 1811+1.6 kb G>T (c. 1679+1643G>T), 1811+1.6 kb A>G (c.1679+1634A>G), 1811+1.6 kb T>A (c. 1679+1650T>A), or W1282X (c.3846G>A) mutation in a CFTR gene in a cell, comprising contacting the cell with one or more splice switching oligonucleotide of claim 1.

29. A method of correcting one or more of a 1811+1.6 kb G>T (c.1679+1643G>T), 1811+1.6 kb A>G (c.1679+1634A>G), 1811+1.6 kb T>A (c.1679+1650T>A), or W1282X (c.3846G>A) mutation in a pre-mRNA produced from a CFTR gene in a subject, comprising administering to the subject an effective amount of the pharmaceutical composition of claim 26, thereby correcting the 1811+1.6 kb G>T (c. 1679+1643G>T), 1811+1.6 kb A>G (c.1679+1634A>G), 1811+1.6 kb T>A (c.1679+1650T>A), or W1282X (c.3846G>A) mutation.

30. A method of treating cystic fibrosis in a subject in need thereof, comprising administering to the subject a therapeutically effective amount of the pharmaceutical composition of claim 26, thereby treating cystic fibrosis in the subject.

31-32. (canceled)

33. The method of claim 30, further comprising administering an oligonucleotide endosomal compound to the subject.

34. (canceled)

35. The method of claim 30, further comprising administering a CFTR modulator compound to the subject.

36. (canceled)
